(19) United States
(12) Patent Application Publication
(10) Pub. No.: US 2004/0266836 A1
(43) Pub. Date: Dec. 30, 2004

(54) Organic compounds

(76) Inventors: Peter Buhlmayer, Arlesheim (CH); Randy Lee Webb, Flemington, NJ (US)

Correspondence Address: NOVARTIS, CORPORATE INTELLECTUAL PROPERTY, ONE HEALTH PLAZA 430/2, EAST HANOVER, NJ, 07936-1080, US

(21) Appl. No.: 10/493,040

(22) PCT Filed: Oct. 17, 2002
(86) PCT No.: PCT/EP02/11652

Related U.S. Application Data
(60) Provisional application No. 60/330,337, filed on Oct. 18, 2001.

(51) Int. Cl. A61K 31/455; C07D 213/46; C07D 211/82

(52) U.S. Cl. 514/355; 546/315

(57) ABSTRACT

The invention relates to a salt formed of at least one AT₁-receptor antagonist having at least one acidic center and of at least one cardiovascular ingredient having at least one basic center that can be used for treating cardiovascular diseases and conditions, their prophylaxis or delay of progression.

This invention relates to salts formed of an AT₁-receptor antagonist and a cardiovascular ingredient, pharmaceutical compositions thereof and methods of using said salts to treat patients suffering from cardiovascular diseases and related conditions.

As cardiovascular diseases are leading causes of death in industrialised countries and as the majority of affected patients are middle aged, there exists an increasing need for potent as well as easily administered drugs for the therapy of cardiovascular diseases. Under the term cardiovascular diseases and related conditions commonly used in the art as a generic term may be summarized diseases or conditions such as e. g. hypertension, congestive heart failure, post myocardial infarction, angina pectoris, coronary heart diseases, cardiac arrhythmia, atherosclerosis, endothelial dysfunction, renal diseases, e.g. acute and preferably chronic renal disease, isolated systolic hypertension, peripheral vascular disease, hyperlipidemia, stroke or end-organ damage. Although drug classes as well as their combinations for the treatment of said diseases and related conditions are well known and commonly used, the patient compliance is often reduced due to the complex therapeutic schemes and the high intake rates.

The present invention provides a unique concept, which is an alternative to fixed combinations. For the first time, combination salts of at least one AT₁ receptor antagonist with at least one further basic cardiovascular active principle (ingredient) have been made. In a fixed combination, different active principles are separately combined in a dosage unit form. In a conventional fixed combination, acidic or basic active ingredients may react within the dosage unit form with pharmaceutical auxiliaries and additives, e.g. by forming esters and the like. A prolonged storage of corresponding dosage unit forms may result in an increased amount of side products. A dosage unit form comprising a combination salt formed of at least two active principles is more stable and resistant to forming unwanted side products. Furthermore, combination salts according to the present invention have improved properties. Corresponding improved properties comprise suitable and improved therapeutic activities and pharmacodynamic effects. For example, the beneficial effects of a combination salt formed of at least two active principles comprises a broader therapeutic applicability, a potentiation or synergism of the activity of one of the salt components, an enhancement of the pharmacodynamic properties. A combination salt can also be used to achieve therapeutic effects even at sub-therapeutic amounts of at least one active principle within a corresponding combination salt.

Accordingly, the invention provides salts formed of at least one AT₁-receptor antagonist having at least one acidic center and of at least one cardiovascular ingredient having at least one basic center.

The term “at least one” whenever referred to herein may define one, two or more units.

The term “acidic center” whenever referred to herein means a functional group able to split of a proton e.g. the hydrogen atom of the carboxyl group, of the sulphonamide group or that of the tetrazole ring of the AT₁-receptor antagonist.

The term “basic center” whenever referred hereinto means a functional group able to gain a proton, for example, the basic amino function in e.g. alkylamines, such as ?? , or any basic heterocycles, such as pyridines, pyrazines, or piperidines.

The term “patient” whenever referred to herein means mammals including humans.

The term “additive effect” or “synergistic effect”, respectively, whenever referred to herein, for example, may be defined using the following equations:

a/A+b/B=1 means an additive effect

a/A+b/B<1 means a synergistic or superadditive effect,

where “A” and “B” are equieffective doses of the individual ingredients when each is present alone; “a” and “b” are the respective doses in the salt according to the invention that give the effect level achieved by dose “A” alone or dose “B” alone. Reference is made to R. J. Tallarida. “Statistical analysis of drug combinations for synergism”, Pain 1992; 49:93-97 and “Drug synergism and dose-effect data analysis”, Editor, R. J. Tallarida, Chapman and Hall/CRC, New York, 2000.

It is expected that in the case of the salts according to the invention, effects will be assessed e.g. as changes in blood pressure and more specifically, as antihypertensive effects. Therefore, the two ingredients forming a salt according to the invention would be synergistic when their effects are greater than that expected from the sum of the individual ingredients, especially at the same doses as in the salt. The hypothesis to be tested is that the two active ingredients are additive when given together. If they are not additive, then an interaction has occurred that may result e.g. in a superadditive response. With a careful experimental design, this can be assessed using appropriate statistical methods as referenced above, e.g. corresponding effects can be taken from dose-response-curves.

In addition to blood pressure, the measurements of heart rate or neurohormones (plasma renin activity, aldosterone or norepinephrine) may also be used to determine the relationship between the salt and their individual component drugs.

Finally, the determination of plasma levels of each component of the salt can also be used to indicate a response, for example, additive or synergistic, that is different from that of the component parts. It is understood that a direct relationship exists between the amount of drug in the plasma and the pharmacologic effect induced by the drug. Therefore, drug levels of the component drugs contained within the salt can be used, either alone or in conjunction with the pharmacodynamic response to demonstrate an advantage of the salt formulation over that of individual components of the salt.

The term “potentiation” shall mean an enhancement of a corresponding pharmacological activity or therapeutic effect, respectively. Potentiation of one component within the combination salt according to the present invention formed with another component within the combination salt according to the present invention means that an effect is being achieved that is greater than that achieved with one component alone.

The term “synergistic effect” or “effect of “potentiation” is used in the context of this application to mean an enhanced responsiveness to the drug. For example, if the salts according to the invention result in an improvement in the absorption, with an increase in the plasma concentration of the AT₁-receptor blocker then the extent of e.g. blood pressure lowering may be “potentiated” or greater than expected.

The pharmacodynamic properties of one active principle within the combination salt of the present invention can also be potentiated by the further active principle within the combination salt of the present invention leading to synergism or potentiation.

The term “AT₁-receptor antagonist” (also called Angiotensin-II-receptor antagonist) whenever referred hereinto has to be understood to define those active ingredients that bind to the AT₁-receptor subtype of Angiotensin-II but do not result in an activation of the receptor and prevent activation of the receptor by Angiotensin-II.

The term “cardiovascular ingredient” whenever referred hereinto means an ingredient selected from the group consisting of calcium channel blockers, endothelin antagonists, renin inhibitors, beta-blockers, alpha-adrenergic antagonists and alpha-adrenergic agonists.

The class of AT₁-receptor antagonist comprises compounds having differing structural features, essentially preferred are the non-peptidic ones. For example, mention may be made of the compounds that are selected from the group consisting of valsartan which is disclosed in EP 443 983, losartan disclosed in EP 253 310, candesartan disclosed in EP 459 136, eprosartan disclosed in EP 403 159, irbesartan disclosed in EP 454 511, olmesartan disclosed in EP 503 785, tasosartan disclosed in EP 539 086 and telmisartan disclosed in EP 522 314, all of which are incorporated herein by reference. Preferred AT₁-receptor antagonists are those agents that have been marketed, most preferred is valsartan.

Calcium channel blockers (CCBs) are known to those skilled in the art. Examples of CCBs useful for the salts of the invention are preferably dihydropyridine representatives selected from the group consisting of amlodipine, nitrendipine, nimodipine, nicardipine, nisoldipine, felodipine, isradipine and nifedipine. Preferred calcium channel blockers are those agents that have been marketed, most preferred is amlodipine disclosed in EP 89167, especially the besylate or maleate thereof.

Endothelin antagonists are highly specific competitive inhibitors of endothelin and modifications of the molecule itself. The endothelin receptors can be subdivided in type A and type B. Most of the endothelin antagonists have an affinity for both receptor types. An example of an endothelin antagonist useful for the salts of the invention is preferably tezosentan.

Renin inhibitors have enzyme-inhibiting properties and inhibit in particular the action of the natural enzyme renin. As a result a smaller amount of Angiotensin-II is produced whose reduced concentration is the direct cause of the antihypertensive effect of renin inhibitors. An example of a renin inhibitor useful for the salts of the invention is preferably (2S,4S,5S,7S)-5-amino4-hydroxy-2-isopropyl-7-[4-methoxy-3-(3-methoxy-propoxy)-benzyl]-8-methyl-nonansäure (2-carbamoyl-2-methyl-propyl)-amid disclosed in EP 678503 and incorporated herein by reference. Especially preferred is the hemi-fumarate salt thereof.

Beta-blockers are known in the art. A beta blocker preferably is a representative selected from the group consisting of a selective β1-blocker, such as atenolol, bisoprolol (especially the fumarate thereof), metoprolol (especially the hemi-(R,R)fumarate or fumarate thereof), furthermore, acebutolol (especially the hydrochloride thereof), esmolol (especially the hydrochloride thereof), celiproplol (especially the hydrochloride thereof), taliprolol, or acebutolol (especially the hydrochloride thereof), a non-selective β-blocker, such as oxprenolol (especially the hydrochloride thereof), pindolol, furthermore, propanolol (especially the hydrochloride thereof), bupranolol (especially the hydrochloride thereof), penbutolol (especially the sulphate thereof), mepindolol (especially the sulphate thereof), carteolol (especially the hydrochloride thereof) or nadolol, and a β-blocker with α-blocking activity such as carvedilol; or in each case, a pharmaceutically acceptable salt thereof. Preferred examples of beta-blockers useful for the salts of the invention are especially beta-blockers that have been marketed preferably carvedilol, oxprenolol, propanolol and metoprolol. Most preferred is oxprenolol.

Alpha-adrenergic antagonists are known in the art. Examples of alpha-adrenergic antagonists useful for the salts of the invention are preferably terazosin disclosed in U.S. Pat. No. 4,026,894 and metazosin disclosed in U.S. Pat. No. 4,775,673.

Alpha-adrenergic agonists are known in the art. An example of an alpha-adrenergic agonist useful for the salts of the invention is preferably moxonidine disclosed in U.S. Pat. No. 4,323,570.

The structure of the active agents identified by generic names may be taken from the actual edition of the standard compendium “The Merck Index” or from databases, e.g. (LifeCycle) Patents International (e.g. IMS world Publications). The corresponding content thereof is hereby incorporated by reference. Any person skilled in the art is fully enabled to identify the active agents and, based on these references, likewise enabled to manufacture and test the pharmaceutical indications and properties e.g. in standard test models, both in vitro and in vivo.

Representative studies are carried out with a salt formed of valsartan and amlodipine, e.g. applying the following methodology. All experiments are performed in spontaneously hypertensive rats (SHR) supplied by Taconic Farms, Germantown, N.Y. (Tac:N(SHR)fBR). A radiotelemetric device (Data Sciences International, Inc., St. Paul, Minn.) is implanted into the lower abdominal aorta of all test animals between the ages of 14 to 16 weeks of age. All SHR are allowed to recover from the surgical implantation procedure for at least 2 weeks prior to the initiation of the experiments. The radiotransmitter is fastened ventrally to the musculature of the inner abdominal wall with a silk suture to prevent movement. Cardiovascular parameters are continuously monitored via the radiotransmitter and transmitted to a receiver where the digitized signal is then collected and stored using a computerized data acquisition system. Blood pressure (mean arterial, systolic and diastolic pressure) and heart rate are monitored in conscious, freely moving and undisturbed SHR in their home cages. The arterial blood pressure and heart rate are measured every 10 minutes for 10 seconds and recorded. Data reported for each rat represent the mean values averaged over a 24 hours period and are made up of the 144-10 minute samples collected each day. The baseline values for blood pressure and heart rate consist of the average of three consecutive 24 hours readings taken prior to initiating the drug treatments. All rats are individually housed in a temperature and humidity controlled room and are maintained on a 12 hour light/dark cycle. See also, Webb et al., J. Hypertens. 1998; 16(6): 843-52.

Drugs are administered either at a single dose by oral gavage or with repeated daily administration. Oral dosages of the individual agents may vary but typically will be in the range of for example: valsartan (0.3-30.0 mg/kg/day); amlodipine (1-10 mg/kg/day); renin inhibitor (10-100 mg/kg/day); other agents will be given at appropriate concentrations according to their known pharmacological properties and will often be tested at sub-maximally effective dosages in order to observe any expected potentiation of response. For example, the degree to which blood pressure can be reduced is a function of the extent to which various physiological control systems are activated but is ultimately limited by homeostatic mechanisms that protect against shock. Agents given as the monotherapy are administered at less than maximally effective dosages. Consequently, when salts according to the invention formed of at least one AT₁-receptor antagonist and for example, of at least one CCB are given, a further antihypertensive effect can be achieved. The potential for an enhancement of the anthypertensive effect or a synergistic action can then be demonstrated. Furthermore, synergy can be observed as actions other than those on blood pressure. Salts according to the invention with distinctive mechanisms of action have complementary effects. For example, alpha adrenergic antagonists and CCBs can evoke stimulation of the renin angiotensin or sympathetic nervous systems resulting in an increase in heart rate. When a salt according to the invention is given, blockade of the AT₁-receptor can prevent the activation of compensatory cardiovascular reflexes and thereby attenuate an increase in heart rate. While this beneficial response can occur when both agents are administered as individual components, the effect is greatest when a salt according to the invention is used. A salt according to the invention could result in better absorption of both components, a more rapid absorption and/or a more complementary peak plasma concentration of both components. The overlap of peak plasma concentrations of the AT₁-receptor antagonist and the cardiovascular ingredient would enable the AT₁-receptor blocker to minimize the negative or detrimental effects of the cardiovascular ingredient.

Following either single or repeated administration of the test substances, the animal are sacrificed and tissues removed for further analysis. For example, to assess endothelial function, blood vessels can be removed and vasoconstriction and/or vasodilation to various substances can be determined. Typical methods are described by Shetty et al., Biochem. Biophys. Res. Commun. 1993; 191(2): 459-64.

Effects on end-organ damage can best be observed with repeated, chronic dosing. Methods are described by Webb et al., Clin. Exp. Hypertens. 1998; 20(7): 775-93. In these types of studies, effects on renal and cardiac function can be monitored. Histological effects on organ structure can also be determined.

The salts according to the invention result not only in a beneficial, especially potentiation or a synergistic, therapeutic effect, but also in additional benefits such as a surprising prolongation of efficacy, a broader rate of response to therapeutic treatment and surprising beneficial effects on diseases and conditions as specified herein. Normal mammalian systems have a strong capacity to buffer or attenuate the antihypertensive effects resulting from interruption of a single blood pressure regulatory system. Thus, the targeting of two systems simultaneously might provide superior blood pressure control. A salt according to the invention would allow multiple blood pressure regulatory systems to be targeted simultaneously. More effective blood pressure control and less activation of detrimental compensatory responses thus result. This can be demonstrated as an attenuation of heart rate, less cardiac stimulation, improved blood flow to organs, and less activation of vasoactive hormones such as angiotensin, norepinephrine and aldosterone. Any of these effects can be additive or synergistic and ultimately preserve organ function more effectively. Further surprising beneficial effects are e.g. reduction of side effects compared to a monotherapy applying only one of the pharmaceutically active compounds used in the salts disclosed herein.

The good physicochemical properties of the salts according to the invention give the possibility of attaining economic advantages by enabling simpler process steps to be carried out during working up. This is also of great importance to the quality of the active substance and its galenic forms during production, storage and administration to the patients. The salts according to the invention show good stability and quality properties also during storage and distribution.

This invention is directed to salts formed of at least one AT₁ receptor antagonist having at least one acidic center and of at least one cardiovascular ingredient having at least one basic center.

This invention is particularly directed to salts where the AT₁ receptor antagonist is valsartan. Further the invention is directed to salts where the cardiovascular ingredients are selected from the group consisting of calcium channel blockers, endothelin antagonists, renin inhibitors, beta-blockers, alpha-adrenergic antagonists and alpha-adrenergic agonists. More specifically, a salt where the cardiovascular ingredient is selected from the group consisting of amlodipine, oxprenolol and (2S,4S,5S,7S)-5-amino4hydroxy-2-isopropyl-7-[4-methoxy-3-(3-methoxy-propoxy)-benzyl]-8-methyl-nonansäure (2-carbamoyl-2-methyl-propyl)-amid.

This invention is still more particularly directed to salts formed of valsartan and amlodipine, valsartan and oxprenolol and valsartan and (2S,4S,5S,7S)-5-amino-4-hydroxy-2-isopropyl-7-[4-methoxy-3-(3-methoxy-propoxy)-benzyl]-8-methyl-nonansäure (2-carbamoyl-2-methyl-propyl)-amid.

The molar ratio of the AT₁ receptor antagonist to the cardiovascular ingredient within the combination salts can vary and is dependent on the number of acidic and basic centers. For illustration, if the AT₁ receptor antagonist has two acidic centers and the cardiovascular ingredient has one basic center, the molar ratio of corresponding combination salts will be 1:1 and 2:1.

The molar ratio of the AT₁ receptor antagonist to the cardiovascular ingredient of the salts according to the invention is especially 1:1(, 2:1) or 1:2. E.g. in the case where an AT₁ receptor antagonist such as valsartan having two acidic centers is selected for the formulation of the salts according to the invention the molar ratio of AT₁ receptor antagonist to cardiovascular ingredient can be 1:1 or 1:2.

The salts according to the invention exist in a form selected from the group consisting of a crystalline form, partly crystalline form and polymorphous form. The salts according to the invention exist also in amorphous form.

Additionally the salts according to the invention occur as a solvate e.g. a hydrate. Said solvates and hydrates are also within the scope of this invention.

The combination salts according to the present invention can also be combined with a diuretic. The diuretic is preferably selected from the group consisting of bumetanide, ethacrynic acid, furosemide, torsemide, amiloride, spironolactone, eplerenone, triamterene, chlorothalidone, chlorothiazide, hydrochlorothiazide (HCTZ), hydroflumethiazide, methylchlorothiazide, metolazone, and dichlorphenamide. Diuretics may be understood in three classes: thiazides (e.g., HCTZ), potassium sparing (e.g., triamterene, spironolactone) and “loop” diuretics (e.g., furosemide). The most preferred diuretic for the intended combination is a thiazide diuretic, e.g. hydrochlorothiazide.

The daily dosage of the diuretic to be combined with the combination salt according to the inventive present combination is between 5 and 200 mg, preferably between 5 and 100 mg and more preferably between 5 and 50 mg.

In a variation thereof, the present invention likewise relates to a “kit-of-parts”, for example, in the sense that the components (combination salt and diuretic) to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is more beneficial than the effect that would be obtained by use of only any one of the components.

A further object of the invention is the preparation of the salts according to the invention. Accordingly, one of the components is dissolved in an appropriate reaction inert solvent. In the next step, said solution is added to a solution of the other component and the resulting salt is isolated, e.g. by evaporation or by the addition of a less polar solvent. Specifically, a salt of the invention is prepared e.g. by dissolving the free base of a cardiovascular ingredient in an appropriate reaction inert solvent (e.g. an ether, e.g. tetrahydrofuran, diethylether or dioxan, a nitrile, e.g. acetonitrile, a halogenated hydrocarbon, e.g. chloroform or methylenechloride, an ester, e.g. ethylacetate, an alcohol, e.g. ethanol, methanol or isopropanol or a hydrocarbon, e.g. pentane, hexane or petroleum ether) and adding the resulting solution to a solution of the free acid of the AT₁ receptor antagonist in an appropriate reaction inert solvent. As used herein, the expression “reaction inert solvent” and “inert solvent” refer to a solvent or mixture of solvents which does not interact with starting materials, reagents, intermediates or products in a manner which adversely affects the yield of the desired product. The reaction mixture is stirred vigorously at ambient or elevated temperature. Crystallization is accomplished by the addition of less polar solvents to afford the salts according to the invention.

Accordingly the pKₐ-value of the cardiovascular ingredient useful for the preparation of a salt according to the invention is ≧7.

The invention provides also pharmaceutical compositions comprising a salt according to the invention.

The salts according to the invention may be used e.g. in a therapeutically effective amount, optionally together with a pharmaceutically acceptable carrier, for example with an inorganic or organic, solid or optionally also liquid pharmaceutically acceptable carrier, which is suitable for enteral, e.g. rectal, oral or parenteral administration. Although the preferred mode of administration of the compounds of this invention is oral, they may be administered parenterally (subcutaneous, intramuscular, intravenous, transdermal) as well.

The excipients used may be those known in the art for example as described in Remington's Pharmaceutical Sciences, 19th Ed. and Handbook of Pharmaceutical Excipients, or analogous to those known in the art or new excipients having analogous function to those described in the art.

In so far as the procedures for formulation are not described herein such formulation procedures may for example be known in the art, or analogous to those known in the art. Representative procedures are disclosed in for example, Remington's Pharmaceutical Sciences, 19th Ed., Mack Publ., Co., 1995, H. Sucker et al and L. Lachman et al. The Theory and Practice of Industrial Pharmacy, 3rd Ed, 1986 as well as later editions, the contents of all of which are incorporated herein by reference.

The invention provides also pharmaceutical compositions for the treatment of cardiovascular diseases.

The invention provides further the use of a salt according to the invention in the preparation of a medicament for the prophylaxis, the treatment or delay of progression of cardiovascular diseases and conditions.

In another aspect the invention provides a method of treating cardiovascular diseases and conditions related thereto, their prophylaxis or delay of progression comprising administering to a patient in need thereof an effective amount of a salt according to the invention.

It is a fact that cardiovascular diseases, like most diseases, never remain stable. Cardiovascular diseases, if untreated or improperly treated, will progressively worsen with time. High blood pressure is a surrogate marker for underlying cardiovascular disease. The high blood pressure per se exerts an undue stress on blood vessels throughout the body and over time results in further damage to blood vessels and tissues. Chronic antihypertensive treatment will delay or slow the progression of cardiovascular disease by attenuating the rise in blood pressure. If the blood pressure is not reduced, it will lead to structural adaptation in the vasculature (fibrosis, cell necrosis) with stiffening of blood vessels and ultimately to a reduction in blood flow to the vital organs (kidney, heart, brain). It has been shown that chronic antihypertensive therapy effectively reduces the incidence of stroke, cardiac and renal disease.

It is unlikely that treatment would be initiated in a patient with normal blood pressure. However, a patient that is diagnosed with hypertension can be treated with the intent to reduce the elevated blood pressure. In so doing, this treatment can be considered prophylactic for the prevention of subsequent cardiovascular events such as stroke, heart failure and kidney failure. Therefore, it is important to identify hypertension at an early stage and to begin treatment immediately. Early initiation of treatment in hypertensive patients, at a time point when no cardiovascular damage has yet occurred, will be an effective prophylaxis.

The dosage of active salt administered is dependent on the body weight, age, individual condition, and on the form of administration. The dosage amount necessary to achieve the desired therapeutic effect is within the skill of those who practice in the art having the benefit of the disclosure herein.

It is expected that if the salt results in an improvement in the bioavailability of the substances or in an improved pharmacokinetic pattern between the two components, then a reduced dosage of the components will be required over that of the case when both components are administered separately. For example, the AT₁-receptor blocker, when given as a salt according to the invention, may be administered at a dose that would preferably be considered submaximal if it were given alone. It may be expected that a dosage of 3-10 mg/kg of the AT₁-receptor blocker, e.g. valsartan and a corresponding (equi)molar amount of a CCB, e.g. amlodipine, when given as a salt according to the invention would be required to achieve a significant effect on blood pressure whereas a dosage of 10-30 mg/kg of the AT₁-receptor blocker, e.g. valsartan, would be required when administered separately. It is well known that CCBs have demonstrable dose-related side effects and thus it would be anticipated that less side effects can be observed if a lower dose is given while still maintaining the beneficial pharmacological actions.

Further surprising beneficial effects are e.g. reduction of side effects compared to a monotherapy applying only one of the pharmaceutically active compounds used in the salts disclosed herein.

The following examples illustrate the above-described invention; however, it is not intended to restrict the scope of this invention in any manner.

### WORKING EXAMPLE 1

Salt prepared from one equivalent of the AT₁ receptor antagonist, valsartan, (component A) and two equivalents of the cardiovascular ingredient, the renin inhibitor (2S,4S,5S,7S)5-amino-4-hydroxy-2-isopropyl-7-[4-methoxy-3-(3-methoxy-propoxy)-benzyl]-8-methyl-nonansäure (2-carbamoyl-2-methyl-propyl)-amid, (component B)

536 mg of component B (Fumarat) are dissolved in 60 ml of methylenchloride and transferred into the free base by extraction with 15 ml of 2N aqueous sodium hydroxide. The organic phase is washed with water and brine, dryed over sodium sulfate and evaporated in vacuo. Drying of the residue in high vacuo (room temperature, 30 minutes) renders the free base component B (444 mg, 0.804 mMol, MG 551.8). This material is dissolved in 20 ml methylenchloride and treated with 175 mg of component A (0.402 mMol, MG 435.5). The mixture is stirred at room temperature for two hours and then evaporated to dryness. The residue is dissolved in 6 ml of tetrahydrofuran and the solution is added dropwise to 100 ml of hexane (precipitation). Filtration and drying in high vacuo for 72 hours at room temperature gives the product as a colorless powder. MS (M+H)⁺ 436.2 (A) und 552.3 (B).

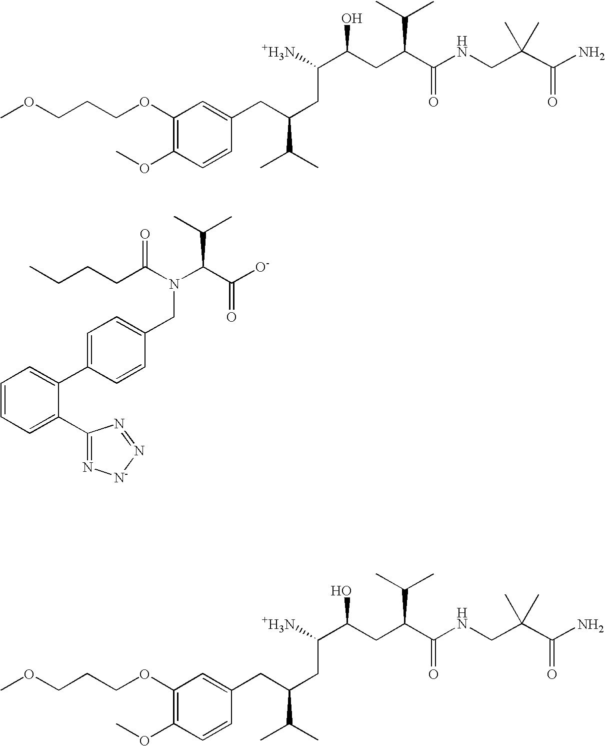

Ratios:

| | Ratio of the components | calculated | Experiment*) |
|---|---|---|---|
| Example 1 | A:B | 1:2 | 1:1.88 |

*)as determined by Area under the curve by HPLC

Elementary analysis (found): C, 65.50; H, 8.48; N, 10.94; O, 15.33; water 0.46.

### WORKING EXAMPLE 2

Salt prepared from one equivalent of component A and one equivalent component B

300 mg of component B (0.544 mMol, MG 551.8) received by extraction of a solution of 350 mg of the fumarate in methylenchloride with 1N aqueous of sodium hydroxide are dissolved in a small amount of ethylacetate and treated with und 237 mg of component A (0.544 mMol, MG 435.5). When complete dissolution is achieved the salt product is precipitated by adding petroleum ether. Ultrasonification and scratching yields homogenized material that can be isolated by filtration. Drying in high vacuo overnight at room temperature gives the product as a white powder. MS (M+H)⁺ 436.2 (A) und 552.3 (B).

Melting point ca. 115° C. (dec.).

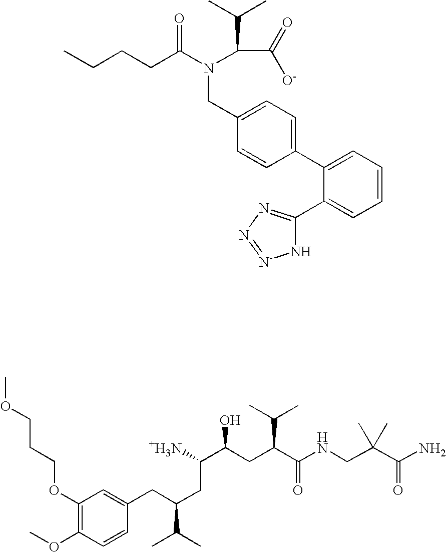

Ratios:

| | Ratio of the components | calculated | Experiment*) |
|---|---|---|---|
| Example 2 | A:B | 1:1 | 1:1.03 |

*)as determined by Area under the curve by HPLC

The following salts can be prepared in a manner as described in Working Examples 1 or 2:

### WORKING EXAMPLES 3-6

Salt prepared from one equivalent of component A and two equivalents of oxprenolol; elementary analysis (found): C, 66.77; H, 7.86; N, 10.01; O, 15.31; water 0.11.

Salt prepared from one equivalent of component A and one equivalent of oxprenolol, elementary analysis (found): C, 66,47; H, 7.59; N, 11.95; O, 14.24; water 0.5.

Salt prepared from one equivalent of component A and two equivalents of amlodipine; elementary analysis (found): C, 60.98; H, 6.45; N, 9.75; Cl, 5.57; O, 17.25; water 0.56.

Salt prepared from one equivalent of component A and one equivalent of amlodipine, elementary analysis (found): C, 62.03; H, 6.56; N, 11.17; Cl, 4.22; O, 15.82; water 0.42.

### FORMULATION EXAMPLE 1

Tablets, each containing 50 mg of a salt of the invention, for example a salt formed of valsartan and (2S,4S,5S,7S)-5-amino-4-hydroxy-2-isopropyl-7-[4-methoxy-3-(3-methoxy-propoxy)-benzyl-8-methyl-nonansäure (2-carbamoyl-2-methyl-propyl)-amid, can be prepared as follows:

Composition (for 10,000 tablets)

| | |
|---|---|
| Active ingredient | 500.0 g |
| Lactose | 500.0 g |
| Potato starch | 352.0 g |
| Gelatin | 8.0 g |
| Talc | 60.0 g |
| Magnesium stearate | 10.0 g |
| Silica (highly disperse) | 20.0 g |
| Ethanol | q.s. |

The salt of the invention is mixed with the lactose and 292 g of potato starch, and the mixture is moistened using an alcoholic solution of the gelatin and granulated by means of a sieve. After drying, the remainder of the potato starch, the talc, the magnesium stearate and the highly disperse silica are admixed and the mixture is compressed to give tablets of weight 145.0 mg each and salt content of 50.0 mg which, if desired, can be provided with breaking notches for finer adjustment of the dose.

### FORMULATION EXAMPLE 2

Coated tablets, each containing 100 mg of a salt of the invention, for example a salt formed of valsartan and (2S,4S,5S,7S)-5-amino-4-hydroxy-2-isopropyl-7-[4-methoxy-3-(3-methoxy-propoxy)-benzyl]-8-methyl-nonansäure (2-carbamoyl-2-methyl-propyl)-amid, can be prepared as follows:

Composition (for 1000 tablets):

| | |
|---|---|
| Active ingredient | 100.00 g |
| Lactose | 100.00 g |
| Corn starch | 70.00 g |
| Talc | |
| Calcium stearate | 1.50 g |
| Hydroxypropylmethylcellulose | 2.36 g |
| Shellac | 0.64 g |
| Water | q.s. |
| Dichloromethane | q.s. |

The salt of the invention, the lactose and 40 g of the corn starch are mixed and moistened and granulated with a paste prepared from 15 g of corn starch and water (with warming). The granules are dried, and the remainder of the corn starch, the talc and the calcium stearate are added and mixed with the granules. The mixture is compressed to give tablets (weight: 280 mg) and these are coated with a solution of the hydroxypropylmethylcellulose and the shellac in dichloromethane (final weight of the coated tablet: 283 mg).

### FORMULATION EXAMPLE 3

Tablets and coated tablets containing a salt of the invention, for example as in one of Working Examples 1-6, can also be prepared in an analogous manner to that described in Formulation Examples 1 and 2.

1. A salt formed of at least one AT₁-receptor antagonist having at least one acidic center and of at least one cardiovascular ingredient having at least one basic center.

2. A salt according to claim 1 where the AT₁-receptor antagonist is valsartan.

3. A salt according to claim 1 where the cardiovascular ingredient is selected from the group consisting of calcium channel blockers, endothelin antagonists, renin inhibitors, beta-blockers, alpha-adrenergic antagonists and alpha-adrenergic agonists.

4. A salt according to claim 1 where the cardiovascular ingredient is selected from the group consisting of amlodipine, oxprenolol and (2S,4S,5S,7S)-5-amino-4-hydroxy-2-isopropyl-7-[4-methoxy-3-(3-methoxy-propoxy)-benzyl]-8-methyl-nonansäure (2-carbamoyl-2-methyl-propyl)-amid.

5. A salt according to claim 4 selected from the salts formed of valsartan and amlodipine, valsartan and oxprenolol and valsartan and (2S,4S,5S,7S)-5-amino-4-hydroxy-2-isopropyl-7-[4-methoxy-3-(3-methoxy-propoxy)-benzyl]-8-methyl-nonansäure (2-carbamoyl-2-methyl-propyl)-amid.

6. A salt according to claim 5 where the molar ratio of AT₁-receptor antagonist to cardiovascular ingredient is 1:1 or 1:2.

7. A salt according to claim 1 in a form selected from the group consisting of a crystalline form, partly crystalline form, amorphous form and polymorphous form.

8. A salt according to claim 7 which is in the form of a solvate or hydrate.

9. A process for the manufacture of a salt according to claim 1 characterized in that a solution of an AT₁-receptor antagonist is mixed with a solution of a cardiovascular ingredient and the resulting salt is isolated.

10. A pharmaceutical composition comprising a salt according to claim 1 and a pharmaceutically acceptable carrier.

11. A method of treating cardiovascular diseases and conditions, their prophylaxis or delay of progression comprising administering to a patient in need thereof an effective amount of a salt according to claim 1.

12. Use according to claim 11 for the prophylaxis, treatment or delay of progression of cardiovascular diseases and conditions selected from the group consisting of hypertension, congestive heart failure, post myocardial infarction, angina pectoris, coronary heart diseases, cardiac arrhythmia, atherosclerosis, endothelial dysfunction, renal disease, acute renal disease, chronic renal disease, isolated systolic hypertension, peripheral vascular disease, hyperlipidemia, stroke and end-organ damage.

13. A salt according to claim 2 where the cardiovascular ingredient is selected from the group consisting of amlodipine, oxprenolol and (2S,4S,5S,7S)-5amino4-hydroxy-2-isopropyl-7-[4-methoxy-3-(3-methoxy-propoxy)-benzyl]-8-methyl-nonansäure (2-carbamoyl-2-methyl-propyl)-amid.

14. A salt according to claim 3 where the cardiovascular ingredient is selected from the group consisting of amlodipine, oxprenolol and (2S,4S,5S,7S)-5-amino-4-hydroxy-2-isopropyl-7-[4-methoxy-3-(3-methoxy-propoxy)-benzyl]-8-methyl-nonansäure (2-carbamoyl-2-methyl-propyl)-amid.
